(19) United States
(12) Patent Application Publication
(10) Pub. No.: US 2006/0025483 A1
(43) Pub. Date: Feb. 2, 2006

(54) (-)-Hydroxycitric acid for protection against soft tissue and arterial calcification

(76) Inventor: Dallas L. Clouatre, Santa Monica, CA (US)

Correspondence Address: DALLAS L. CLOUATRE, 1223 WILSHIRE BLVD #761, SANTA MONICA, CA, 90403-5400, US

(21) Appl. No.: 11/174,910

(22) Filed: Jul. 5, 2005

Related U.S. Application Data
(60) Provisional application No. 60/599,222, filed on Jul. 29, 2004.

(51) Int. Cl. A61K 31/19

(52) U.S. Cl. 514/574

(57) ABSTRACT

The inventor has discovered that supplementation with (−)-hydroxycitric acid, its salts and related compounds constitutes a novel means of inhibiting, reducing and regulating calcification of the blood vessels and other soft tissues and is useful for preventing, treating and ameliorating conditions involving soft tissue calcification. Such regulation offers benefits against arterial calcification and vascular diseases, osteoarthritis, rheumatoid arthritis, the calcification of surgical stints, such as those containing elastin. These benefits of HCA are especially pronounced with the use of the preferred salts of the acid, potassium hydroxycitrate and potassium-magnesium hydroxycitrate, and may be further potentiated by the use of a controlled-release form of the compound. The discovery that HCA has calcium-regulating effects in the soft tissues allows for the creation of novel and more efficacious approaches to preventing and ameliorating cardiovascular diseases, arthritis and a variety of other conditions. Inasmuch as one element common to advancing years is an increased level of generalized calcification of the soft tissues, the invention lends itself to reducing or delaying this aspect of aging. Furthermore, this discovery makes possible the development of adjuvant modalities that can be used to improve the results realized with other treatment compounds while at the same time reducing the side effects normally found with such drugs. HCA delivered in the form of its potassium salt is efficacious at a daily dosage (bid or tid) of between 750 mg and 10 grams, preferably at a dosage of between 3 and 6 grams for most individuals. A daily dosage above 10 grams might prove desirable under some circumstances, such as with extremely large or resistant individuals, but this level of intake is not deemed necessary under normal conditions.

### PROVISIONAL PATENT APPLICATION FILING

Entitled to the benefit of Provisional Patent Application Ser. No. 60/599,222 filed Jul. 29, 2004, “(−)-Hydroxycitric Acid For Protection Against Soft Tissue And Arterial Calcification.”

### BACKGROUND OF THE INVENTION

1. Field Of The Invention

This invention relates to pharmaceutical compositions containing (−)-hydroxycitric acid, its salts and related compounds useful for reducing and regulating calcification of the blood vessels and other soft tissues. Such regulation offers benefits against arterial calcification and vascular diseases, osteoarthritis, rheumatoid arthritis, and the calcification of surgical stints, such as those containing elastin.

2. Description Of Prior Art

(−)-Hydroxycitric acid (abbreviated herein as HCA), a naturally-occurring substance found chiefly in fruits of the species of Garcinia, and several synthetic derivatives of citric acid have been investigated extensively in regard to their ability to inhibit the production of fatty acids from carbohydrates, to suppress appetite, and to inhibit weight gain. (Sullivan A C, Triscari J. Metabolic regulation as a control for lipid disorders. I. Influence of (−)-hydroxycitrate on experimentally induced obesity in the rodent American Journal of Clinical Nutrition 1977;30:767-775.) Weight loss benefits were first ascribed to HCA, its salts and its lactone in U.S. Pat. No. 3,764,692 granted to John M. Lowenstein in 1973. The claimed mechanisms of action for HCA, most of which were originally put forth by researchers at the pharmaceutical firm of Hoffmann-La Roche, have been summarized in at least two United States Patents. In U.S. Pat. No. 5,626,849 these mechanisms are given as follows: “(−) HCA reduces the conversion of carbohydrate calories into fats. It does this by inhibiting the actions of ATP-citrate lyase, the enzyme that converts citrate into fatty acids and cholesterol in the primary pathway of fat synthesis in the body. The actions of (−) HCA increase the production and storage of glycogen (which is found in the liver, small intestine and muscles of mammals) while reducing both appetite and weight gain. (−) Hydroxycitric acid also causes calories to be burned in an energy cycle similar to thermogenesis . . . (−) HCA also increases the clearance of LDL cholesterol . . . ” U.S. Pat. No. 5,783,603 further argues that HCA serves to disinhibit the metabolic breakdown and oxidation of stored fat for fuel via its effects upon the compound malonyl CoA and that gluconeogenesis takes place as a result of this action. The position that HCA acts to unleash fatty acid oxidation by negating the effects of malonyl CoA with gluconeogenesis as a consequence (McCarty M F. Promotion of hepatic lipid oxidation and gluconeogenesis as a strategy for appetite control. Medical Hypotheses 1994;42:215-225) is maintained in U.S. Pat. No. 5,914,326.

Most of the primary research on HCA was carried out by Hoffman-La Roche nearly three decades ago. The conclusion of the Roche researchers was that “no significant differences in plasma levels of glucose, insulin, or free fatty acids were detected in (−)-hydroxycitrate-treated rats relative to controls. These data suggest that peripheral metabolism, defined in the present context as metabolite flux, may be involved in appetite regulation . . . ” (Sullivan, Ann C. and Joseph Triscari. Possible interrelationship between metabolite flux and appetite. In D. Novin, W. Wyriwicka and G. Bray, eds., Hunger: Basic Mechanisms and Clinical Implications (New York: Raven Press,1976) 115-125.)

HCA is highly researched as of 2005, with 157 citations appearing on PubMed under “hydroxycitrate” and 101 appearing under “hydroxycitric acid.” Quite surprisingly, HCA has been discovered by the inventor to regulate calcification of the soft tissues. Such regulation offers benefits against arterial calcification and vascular diseases, osteoarthritis, rheumatoid arthritis, and the calcification of surgical stints, such as those containing elastin. No existing literature teaches such a role despite more than three decades of active research on the compound. The inventor's claims regarding HCA clearly are novel.

Unlike most serum lipid markers, which unless they are oxidized primarily are putative indicators of cardiovascular disease risk rather than causal agents, now that proper measurement techniques have been developed, it has been shown that vascular calcification is a highly significant factor in the initiation, progression and physiologic actions of arterial plaques. Indeed, the preponderance of available evidence indicates that uncalcified plaques are relatively benign. In addition, inhibition of calcification effectively inhibits the plaque formation process without any alteration in serum cholesterol levels, something demonstrated conclusively thirty years ago. (Chan C T, Wells H, Kramsch D M. Suppression of calcific fibrous-fatty plaque formation in rabbits by agents not affecting elevated serum cholesterol levels. The effect of thiophene compounds. Circ Res. 1978 July;43(1): 115-25.) These results are reproducible with other compounds that are calcium inhibitors. (Sugano M, Nakashima Y, Tasaki H, Takasugi M, Kuroiwa A, Koide O. Effects of diltiazem on suppression and regression of experimental atherosclerosis. Br J Exp Pathol. 1988 August;69(4):515-23.) The challenge, of course, is to find calcium agonists that act only locally in the vascular system without negatively influencing bone mineralization or other health parameters.

Calcification, in any event, is highly correlated with carotid and arotic wall changes. For instance, the results of the Rotterdam Coronary Calcification Study, a recent population-based study in subjects age 55 years and over. Participants of the study underwent an electron beam CT scan. Coronary calcification was quantified according to the Agatston calcium score. Measures of extracoronary atherosclerosis included common carotid intima media thickness (IMT), carotid plaques, ankle-arm index (AAI) and aortic calcification. The first 2,013 participants were used for the present analyses. Age-adjusted geometric mean calcium scores were computed for categories of extracoronary measures using analyses of variance. Graded associations with coronary calcification were found for the carotid and aortic measures. Associations were strongest for carotid plaques and aortic calcification; coronary calcification increased from the lowest category (no plaques) to the highest category 9-fold and 11-fold in men and 10-fold and 20-fold in women, respectively. A nonlinear association was found for AAI with an increase in coronary calcification only at lower levels of AAI. (Oei H H, Vliegenthart R, Hak A E, Iglesias del Sol A, Hofman A, Oudkerk M, Witteman J C. The association between coronary calcification assessed by electron beam computed tomography and measures of extracoronary atherosclerosis: the Rotterdam Coronary Calcification Study. J Am Coll Cardiol. 2002 June 5;39(11):1745-51.) Moreover, calcification, which is an active component of direct damage to the cardiovascular system, is much more sensitive than are the so-called risk factors. Almost 30% of the men and 15% of the women without risk factors examined in the Rotterdam Study had extensive coronary calcification. (Oei H H, Vliegenthart R, Hofman A, Oudkerk M, Witteman J C. Risk factors for coronary calcification in older subjects. The Rotterdam Coronary Calcification Study. Eur Heart J. 2004 January;25(1):48-55.) Calcification is highly predictive of myocardial infarctions. (Vliegenthart R, Oudkerk M, Song B, van der Kulp D A, Hofman A, Witteman J C. Coronary calcification detected by electron-beam computed tomography and myocardial infarction. The Rotterdam Coronary Calcification Study. Eur Heart J. 2002 October;23(20):1596-1603.) Calcification, similarly, is predictive of stroke. (Vliegenthart R, Hollander M, Breteler M M, van der Kuip D A, Hofman A, Oudkerk M, Witteman J C. Stroke is associated with coronary calcification as detected by electron-beam CT: the Rotterdam Coronary Calcification Study. Stroke. 2002 February;33(2):462-5.) Similarities in the pathogenesis of arterial and articular cartilage calcification have come to light in recent years. These include the roles of aging, of chronic low-grade inflammation and so forth and so on. (Rutsch F, Terkeltaub R Deficiencies of physiologic calcification inhibitors and low-grade inflammation in arterial calcification: lessons for cartilage calcification. Joint Bone Spine. 2005 March;72(2):110-8.) As another example, matrix metalloproteinase-9 (MMP-9), accepted as a primary actor in vascular calcification, has been demonstrated to be active in arthritis and joint diseases. (Itoh T, Matsuda H, Tanioka M, Kuwabara K, Itohara S, Suzuki R. The role of matrix metalloproteinase-2 and matrix metalloproteinase-9 in antibody-induced arthritis. J Immunol. 2002 September 1;169(5):2643-7.) Kidney disease/end stage renal failure is similarly plagued by tissue calcification, which usually is attributed to altered serum calcium and phosphate balances, yet can be given an alternative analysis not prejudicial to the phosphate balance hypothesis. It can be shown that factors, such as angiotensin-converting enzyme, that influence the progression of renal failure also play a direct role in vascular calcification. (Chiurchiu C, Remuzzi G, Ruggenenti P. Angiotensin-converting enzyme inhibition and renal protection in nondiabetic patients: the data of the meta-analyses. J Am Soc Nephrol. 2005 March;16 Suppl 1:S58-63.) Both direct and indirect mechanisms are in common between vascular and a number of other forms of soft tissue calcification. Moreover, there is a linkage between calcification and other untoward changes in vascular tissues. Experimentally, it has been demonstrated that administration of bisphosphonates decreases not only mineral deposition, but also the accumulation of cholesterol, elastin and collagen in these tissues.

A known influence in vascular calcification is elevated insulin and blood glucose. Hyperglycemia alters metalloproteinase activity and thus acts on a major factor in vascular calcification, perhaps via oxidative stress. (Uemura S, Matsushita H, Li W, Glassford A J, Asagami T, Lee K H, Harrison D G, Tsao P S. Diabetes mellitus enhances vascular matrix metalloproteinase activity: role of oxidative stress. Circ Res. 2001 June 22;88(12):1291-8.) However, as the well-known failures of supplementation with vitamins C and E have demonstrated, merely ingesting antioxidants does not seem to alter the actions of localized and system oxidative stress sufficiently to give significant cardiovascular protection. Similarly, as demonstrated by the actually increased rates of morbidity and mortality found with a number of diabetes drugs, mere regulation of blood sugar levels is not enough. Although there is universal agreement that tight regulation of blood sugar levels should be beneficial, the sulfonylurea class of drugs in terms of end points has proved to be a failure-in various trials, the death rate went up in comparison with blood sugar regulation via diet and exercise alone.

In contrast, diabetes drugs that influence ligands for peroxisome proliferator-activated receptor-γ (PPAR-γ) have beneficial effects on the arterial wall in atherosclerosis, perhaps via an anti-inflammatory mechanism. (Gaillard V, Casellas D, Seguin-Devaux C, Schohn H, Dauca M, Atkinson J, Lartaud I. Pioglitazone Improves Aortic Wall Elasticity in a Rat Model of Elastocalcinotic Arteriosclerosis. Hypertension. 2005 June 20; [Epub ahead of print]) It must be stressed that anti-inflammatory does not necessarily mean anti-oxidant. Moreover, other factors are at work. Pioglitazone has been shown to act independently of simple glycemic control and to positively influence direct regulators of vascular calicification, such as vascular endothelial growth factor, matrix metalloproteinase (MMP-9) and monocyte chemoattractant protein (MCP-1). (Pfutzner A, Marx N, Lubben G, Langenfeld M, Walcher D, Konrad T, Forst T. Improvement of cardiovascular risk markers by pioglitazone is independent from glycemic control: results from the pioneer study. J Am Coll Cardiol. 2005 June 21 ;45(12): 1925-31.) Aside from the actions of hyperinsulinemia and hyperglycemia, conveniently placed under such headings as the Insulin Resistance Syndrome/the Metabolic Syndrome/Syndrome X and covered by our issued U.S. Pat. No. 6,207,714, several other mechanisms have been proposed. It is generally accepted that direct testing of these mechanisms in vivo has remained difficult up to the time of this writing in 2005. Nevertheless, it is well established that a number of physiologic substances actively induce, inhibit and/or participate in soft tissue calcification. Among these are:
angiotension I-converting enzyme (ACE)
glucocorticoids
inflammation/localized oxidative stress
leptin
matrix metalloproteinase (MMP-9)
monocyte chemoattractant protein (MCP-1)
peroxisome proliferator-activated receptor-≢ (PPAR-γ)
resistin
tumor necrosis factor-alpha (TNF-α)

It is the current inventor who has demonstrated the relationship of most of the above factors to the actions of HCA and who holds the relevant issued and pending patents governing angiotension-converting enzyme, gluccocorticoids, inflammation, leptin, PPAR-γ, resistin and TNF-α.

No direct data as of yet is available on HCA and MMP-9 or MCP-1. However, it can be shown that both of these are influenced by other compounds/mechanisms discovered by the inventor. In the case of MMP-9, inflammation is a direct activator and local inhibition of vascular tissue inflammation also reduces MMP-9 activity. (Egi K, Conrad N E, Kwan J, Schulze C, Schulz R, Wildhirt S M. Inhibition of inducible nitric oxide synthase and superoxide production reduces matrix metalloproteinase-9 activity and restores coronary vasomotor function in rat cardiac allografts. Eur J Cardiothorac Surg. 2004 August;26(2):262-9.) (Pfutzner A, Marx N, Lubben G, Langenfeld M, Walcher D, Konrad T, Forst T. Improvement of cardiovascular risk markers by pioglitazone is independent from glycemic control: results from the pioneer study. J Am Coll Cardiol. 2005 June 21;45(12):1925-31.) MCP-1 is similarly regulated by localized inflammation. (Doherty T M, Fitzpatrick L A, Shaheen A, Rajavashisth T B, Detrano R C. Genetic determinants of arterial calcification associated with atherosclerosis. Mayo Clin Proc. 2004 February;79(2):197-210.) Available evidence indicates that MMP-9 and MCP-1, therefore, can be modified by regulators of TNF-α and other inflammatory compounds and also by regulators of PPAR-γ. U.S. patent application 20050032901, “(−)-Hydroxycitric acid for controlling inflammation” by the present inventor addresses the issue of inflammation and further data on TNF-α is found in the Examples below. Regulation of PPAR-γ is found in the inventor's U.S. Pat. No. 6,474,071, “Correcting polymorphic metabolic dysfunction with (−)-hydroxycitric acid.”

Knowledge of the role of ACE in vascular calcification is recent. Inflammatory cells release enzymes (including ACE) that generate angiotensin II. One explanation is that a local positive-feedback mechanism could be established in the vessel wall for oxidative stress, inflammation, and endothelial dysfunction. Angiotensin II also acts as a direct growth factor for vascular smooth muscle cells and can stimulate the local production of metalloproteinases and plasminogen activator inhibitor. This is to say that angiotensin-converting enzyme (ACE) activation and the de novo production of angiotensin II contribute to cardiovascular disease through direct pathological tissue effects. (Dzau V J. Theodore Cooper Lecture: Tissue angiotensin and pathobiology of vascular disease: a unifying hypothesis. Hypertension. 2001 April;37(4):1047-52.) ACE is now seen as actively involved in vascular calcification. (Doherty T M, Fitzpatrick L A, Shaheen A, Rajavashisth T B, Detrano R C. Genetic determinants of arterial calcification associated with atherosclerosis. Mayo Clin Proc. 2004 February;79(2):197-210.) The present inventor has discovered a role for HCA in regulating ACE, for which see Provisional Patent Application Ser. No. 60/599223 and now the full U.S. patent application filed Jun. 14, 2005.

Many other factors have been suggested as promoting vascular calcification, but here it is useful to focus only on four of these, to wit, glucocorticoids, leptin, peroxisome proliferator-activated receptor-γ (PPAR-γ) and resistin. A model of the means by which glucocorticoids enhance vascular calcification has been developed. (Mori K, Shioi A, Jono S, Nishizawa Y, Morii H. Dexamethasone enhances In vitro vascular calcification by promoting osteoblastic differentiation of vascular smooth muscle cells. Arterioscler Thromb Vase Biol. 1999 September; 19(9):2112-8.) Leptin, similarly, has been shown to directly enhance calcification of the vascular cells. Leptin possesses procoagulant and antifibrinolytic properties, and it promotes thrombus and atheroma formation, probably through the leptin receptors by promoting vascular inflammation, proliferation, and calcification, and by increasing oxidative stress. (Parhami F, Tintu Y, Ballard A, Fogelman A M, Demer L L. Leptin enhances the calcification of vascular cells: artery wall as a target of leptin. Circ Res. 2001 May 11;88(9):954-60.) (Kougias P, Chai H, Lin P H, Yao Q, Lumsden A B, Chen C. Effects of adipocyte-derived cytokines on endothelial functions: implication of vascular disease. J Surg Res. 2005 June 1;126(1):121-9.) That PPAR-γ suppresses early osteogenenic differentiation in the vascular wall has been established. (Vattikuti R, Towler D A. Osteogenic regulation of vascular calcification: an early perspective. Am J Physiol Endocrinol Metab. 2004 May;286(5):E686-96.) As discussed above, one regulator of PPAR-γ, pioglitazone, has been shown to inhibit arterial calcification. Finally, resistin increases the expression of the adhesion molecules, up-regulates the monocyte chemoattractant chemokine-1 (hence, MCP-1) and promotes endothelial cell activation, hence is a potent activator of vascular calcification. (Kougias P, Chai H, Lin P H, Yao Q, Lumsden A B, Chen C. Effects of adipocyte-derived cytokines on endothelial functions: implication of vascular disease. J Surg Res. 2005 June 1;126(1):121-9.) The modulation of all four of these compounds-glucocorticoids, leptin, peroxisome proliferator-activated receptors (PPAR-γ) and resistin is found in the inventor's U.S. Pat. No. 6,474,071, “Correcting polymorphic metabolic dysfunction with (−)-hydroxycitric acid.”

The period of active research and publication on HCA began in 1969. Until now, it had never been suggested that HCA regulates calcification of the soft tissues and such a claim would appear quite surprising in light of existing publications. Indeed, all of the primary research that supports such a finding has come from the present inventor. Hence, the inventor's claims regarding HCA and the regulation of calcification of vascular and other soft tissues clearly are novel. Regulation offers benefits against arterial calcification and vascular diseases, osteoarthritis, rheumatoid arthritis, and the calcification of surgical stints, such as those containing elastin.

### SUMMARY OF THE INVENTION

The inventor has discovered that supplementation with (−)-hydroxycitric acid, its salts and related compounds is useful for reducing and regulating calcification of the blood vessels and other soft tissues. Such regulation offers benefits against arterial calcification and vascular diseases, osteoarthritis, rheumatoid arthritis, the calcification of surgical stints, such as those containing elastin. These benefits of HCA are especially pronounced with the use of the preferred salts of the acid, potassium hydroxycitrate and potassium-magnesium hydroxycitrate, and may be further potentiated by the use of a controlled-release form of the compound. The discovery that HCA has calcium-regulating effects in the soft tissues allows for the creation of novel and more efficacious approaches to preventing and ameliorating cardiovascular diseases, arthritis and a variety of other conditions. Inasmuch as one element common to advancing years is an increased level of generalized calcification of the soft tissues, the invention lends itself to reducing or delaying this aspect of aging. Furthermore, this discovery makes possible the development of adjuvant modalities that can be used to improve the results realized with other treatment compounds while at the same time reducing the side effects normally found with such drugs. HCA delivered in the form of its potassium salt is efficacious at a daily dosage (bid or tid) of between 750 mg and 10 grams, preferably at a dosage of between 3 and 6 grams for most individuals. A daily dosage above 10 grams might prove desirable under some circumstances, such as with extremely large or resistant individuals, but this level of intake is not deemed necessary under normal conditions.

### OBJECTS AND ADVANTAGES

It is an objective of the present invention to provide a method for preventing, treating or ameliorating conditions that involve calcium deposition in vascular and other soft tissues. These include cardiovascular diseases in general, aortic and other forms of vascular calcification, osteoarthritis, rheumatoid arthritis and calcification of surgical stints. Very few compounds are known that have any reliable effect in these areas and these compounds typically are associated with a variety of side effects. For instance, other PPAR- Y modifiers cause weight gain and statin drugs, which are weak as inhibitors of calcification, are noted for such numerous and unpleasant side effects that approximately seventy-five percent of patients discontinue use within two years. Knowledge of the present invention has the further advantage of allowing the use of forms of (−)-hydroxycitric acid, including especially through controlled release formulations, as adjuvants to cardiovascular drugs and other drugs. In the well established problem of drugs such as warfarin actually promoting vascular calcification, HCA can be employed to ameliorate this side effect.

### DESCRIPTION OF THE PREFERRED EMBODIMENTS

The free acid form and various salts of (−)-hydroxycitric acid (calcium, magnesium, potassium, sodium and mixtures of these) have been available commercially for several years. Any of these materials can be used to fulfill the invention revealed here, but with varying degrees of success. These materials are generally useful in this descending order of efficacy: potassium salt, sodium salt, free acid, magnesium salt, and calcium salt. Exact dosing will depend upon the form of HCA used, the weight of the individual involved, and the other components of the diet. Controlled release can also be expected to improve results by aiding in maintaining a sustained exposure to the drug as required for therapy. The previously patented hydroxycitric acid derivatives (mostly amides and esters of hydroxycititric acid, the patents for which are now expired, to wit, U.S. Pat. Nos. 3,993,668; 3,919,254; and 3,767,678) likely are roughly equivalent to the HCA sodium salt in efficacy.

### EXAMPLE 1

### Clinical Evidence for Blood Glucose/Insulin Regulation

A multi-week pilot open clinical weight loss trial with extremely obese patients was planned to gauge the effects of a pouch delivery form of a potassium salt of (−)-hydroxycitrate under the normal circumstances faced in clinical practice with this patient population. Fourteen patients were enrolled, three of whom were diabetics on medications and several others who were suspected of suffering from insulin resistance. The patients ingested 3-4 grams of HCA per day in two divided doses. Aside from being informed that they must eat a carbohydrate-containing meal within one hour of taking the HCA and that they should avoid eating late in the day, they were not instructed to follow any special diet or exercise plan outside their normal habits and no caloric restriction was imposed. This particular form of potassium (−)-hydroxycitrate delivery typically was mixed into water or juice and consumed at mid-morning and mid-afternoon. The delivery was a water-soluble immediate release form. It was a pre-commercial preparation and nearly all of the patients complained regarding the inconvenience and poor taste of the product, albeit there were no other issues of tolerability. A number of patients continued on the program for 6 weeks. However, comparative data was good for only 3 weeks because two of the diagnosed diabetics experienced hypoglycemic reactions. Several other patients experienced good appetite suppression, yet also complained off episodic tiredness at the beginning of the program, a sign of low blood sugar. Two patients subsequently were placed on phentermine. One patient who followed the program for 10 weeks with excellent weight loss (32 pounds over 10 weeks) found that his tendency toward elevated blood sugar was stabilized during the program. This patient returned to his prior experiences of infrequent hypoglycemia roughly one week after he had left the program, something which suggests a carryover effect from the compound. The average weight loss over the 3 week period for these patients was approximately 3 pounds per person per week. The clinical decision was made that potassium (−)-hydroxycitrate in an immediate release format can exercise a strong hypoglycemic effect in diabetics and that it appears to influence blood sugar levels in protodiabetics, as well. At therapeutically effective dosages, HCA probably should be used with diabetic populations only under a physician's care.

The results of this pilot trial cited in U.S. Pat. No. 6,207,714 and using a pre-production material subsequently have been confirmed by a number of published studies using other models. HCA used appropriately ameliorates insulin resistance and reduces elevated blood sugar levels.

### EXAMPLE 2

### Ace Inhibition: Evidence from Blood Pressure Modulation

A known effect of ACE inhibitors is a reduction in elevated systolic blood pressure. To test this, the following protocol was employed: Sprague-Dawley Rats (SD), approximately 8 weeks of age were obtained. Six groups of eight male SD received the same standard rat chow manufactured to specifications. The special diets derived 30% of calories from fats (one half from lard and one half from corn oil), 50% from carbohydrates, and 20% from proteins. Twenty percent of dietary calories was derived from sucrose and the preponderance of the remaining carbohydrate calories was derived from dextrin. During weekdays (M-F), each group was gavaged twice daily with a solution containing a commercial source of potassium hydroxycitrate (KHCA), a commercial source of potassium-calcium hydroxycitrate (KCaHCA), or a pre-commercial non-salt source of potassium-magnesium hydroxycitrate (KMgHCA, listed as KMgHCA L-Low, M-Intermediate or H-High depending upon the dose). Over the weekends (S-S), a similar quantity of the weekday daily dose was added to twenty grams of food, that is, an amount of food estimated to be close to the daily intake of the animals. At initiation of study and four weeks, and eight weeks later, bloods were drawn from all SD for routine blood chemistries. Body weight (BW) was measured weekly and systolic blood pressure (SBP) was measured every two weeks.

The HCA dosages in the arms varied. The dosage used in the KHCA arm was extrapolated from the recommended 1,500 mg HCA per day for humans consuming a normal diet (i.e., ≧30% calories derived from fats) advocated by a commercial seller of KHCA and claimed to have produced acceptable clinical results. The approximate equivalent for the rat model is 35.4 mg HCA per day, which we increased to 38.4 mg HCA per day for convenience in employing a 48% HCA potassium salt and to remain safely on the high side in practice. For the sake of comparison, a commercial KCaHCA salt (60% HCA) was chosen and delivered at an HCA dosage level of 48 mg per day, which slightly exceeded the lowest dosage of HCA found to be efficacious for inhibition of weight gain in rats in the early pharmaceutical trials (45.4 mg/day) using pure trisodium hydroxycitrate and a very low fat diet. The design thus utilized a realistic diet with rough equivalents of the HCA dosages claimed to be effective in both the human and rat models.

Calculations were based on the early work on HCA by Roche in which the lowest dose in rats shown to be efficacious in reducing weight gain was 0.33 mmol/kg twice a day (delivered as trisodium hydroxycitrate) on a diet consisting of 70% glucose and 1% fat [8]. (−)-Hydroxycitric acid (C₆H₈O₈) has a molecular weight of 208, therefore 1 millimole=208 mg. The rat dose thus would be calculated as 0.33 mmol/kg b.i.d., meaning 208×0.33 kg rat wt (in kg assuming an average weight of 333 grams)=22.65/1000=22.7 mg b.i.d. or 45.4 mg HCA total intake per day, which is equivalent to 76 mg daily of a 60% HCA salt. This should be put in perspective as to the likely lowest efficacious human dose under similar conditions of less than 10% calories from fat in the diet. At 0.33 mmol HCA b.i.d., the human dosage is 208 mg×0.33×70 kg=4.8 grams of HCA per dose×2=9.6 grams HCA/day=16 grams of a 60% salt. Using the normal rat-to-human multiplier for calculating the small animal effect [9], an appropriate dose for humans would be close to 9.6÷5=1.92 grams hydroxycitric acid content on an extremely low fat diet and assuming the material is supplied via a salt that is equivalent to pure trisodium hydroxycitrate in efficacy and is delivered without food effect on uptake.

The experimental KMgHCA dosings varied considerably from that of the other two salts. Subsequent to the start of the trials, it was discovered that the KMgHCA was diluted with as much as 15% potassium chloride (inactive) and that there was a mistake in the calculation of the waters of hydration. As a result, the recalculated HCA doses for the experimental compound were a low dose (KMgHCA L) of 14 mg, an intermediate dose (KMgHCA M) of 28 mg and a high dose (KMgHCA H) of 84 mg per day. The difficulty in calculating the HCA content in this case is not unique inasmuch as there is as of yet no universally accepted method for calculating the HCA content of the various salts. Again, preparations yielded the equivalent of 48 mg HCA per day from KCaHCA and 38.4 mg HCA per day from KHCA.

Systolic Blood Pressure (SBP): SBP was estimated by tail plethysmography in unanesthetized rats after a brief warming period. Readings were taken approximately one minute apart. To be accepted, SBP measurements had to be virtually stable for a minimum of three consecutive readings.

Statistical Analyses: Results are presented as mean±SEM. Many statistics were performed by one-way analysis of variance (ANOVA). SBP and BW were examined by two-way analyses of variance (one factor being dietary group and the second factor being time of examination). Where a significant effect of diet was detected by ANOVA (p<0.05), the Dunnett t test was used to establish which differences between means reached statistical significance (p<0.05). If a Student's t test was employed, this is noted.

Findings for Systolic Blood Pressure: The general trend was for all test groups to consistently show significantly lower SBP during the course of study. The only exception was low-dose of KMgHCA (KMgHCA L), which apparently was below the threshold for effect (FIG. 1). At the end of eight weeks, the doses of the KHCA and KCaHCA and the two higher doses of the KMgHCA caused significant decreases in SBP compared to control (FIG. 2). With regard to 3 different doses of KMgHCA (FIG. 6), the low dose essentially did nothing, but the intermediate and high doses caused virtually the same significant lowering of SBP at the end of 8 weeks—over 10 mm Hg.

Findings for Blood Chemistries: Blood chemistries were obtained at baseline, one month and two months. No significant differences were seen in BUN, and serum creatinine, ALT, AST, and glucose among the six groups. Accordingly, no evidence of liver and renal toxicities was apparent. Although the average insulin concentrations were lower in all KMgHCA groups and in the KHCA group (FIG. 3), the differences were not significant compared to control using ANOVA. The lack of significance may be due to the small numbers of animals examined and the large variances found, especially with control. Only the KCaHCA group did not show a trend toward lower circulating insulin. Recalculating control versus KHCA alone for insulin using the Student's t test showed significance; a similar recalculation of control versus KMgHCA H was at the margin of significance (p=0.058).

An earlier study not described here had demonstrated a decrease in SBP using a KCaHCA salt at a dose of 120 mg HCA per day. In the present study, significantly decreased SBP was produced readily in all the hydroxycitrate groups with the exception of the low dose of KMgHCA (14 mg HCA). One surprising finding was that that the intermediate dose of KMgHCA supplying only 28 mg HCA (KMgHCA M) was equal in this regard to KHCA supplying 38.4 mg HCA and KCAHCA supplying 48 mg HCA (FIG. 2). Another interesting outcome was that elevating the dose of HCA further, in this case to 84 mg in the high KMgHCA dose (KMgHCA H) did not have exert a greater impact on SBP (FIG. 4). Taken together, these findings suggest that there may be a limit to the blood pressure effect of HCA and that this limit is reached with a relatively low dose. Whether all the salts are equally effective remains to be seen. With regard to at least one of the vectors influencing blood pressure, insulin, the KCaHCA salt appears to be significantly less active than the others tested. Moreover, the fact that KCaHCA had little positive impact upon insulin regulation in this model, yet still improved SBP suggests that more than one blood pressure regulating mechanism is at work.

### EXAMPLE 3

### Ace Inhibition: Response to Losarten Challenge

Many factors can positively influence blood pressure, e.g., diuretics, antioxidants, regulators of sympathetic/parasympathetic tone, compounds that improve insulin sensitivity and so forth. Therefore, losartan, an angiotensin-2 receptor blocker, was utilized to discover whether the ACE system was involved in the results discussed in Example 1.

Spontaneously hypertensive rats (SHR) were placed on a diet composed of regular rat chow (60% w/w) and table sugar (40% w/w). This diet reliably elevates blood pressure in this animal model. One group received 100 mg HCA per day in the form of a new potassium-magnesium hydroxycitrate (different from that used in Example 1) via an added 5 g HCA per kg of food mix. Systolic blood pressure and body weight were tested as in Example 1 on a weekly basis.

Over three weeks, there was a trend for an increase in body weight in SHR consuming KMgHCA (p=0.084) in this model. This was viewed as likely positive in that rats gain weight steadily as long as they remain in good health and the SHR at middle age, as used here, lives a relatively short life and its health deteriorates as its blood pressure rises. SBP steadily increased in control as shown in FIG. 5, where delta SBP steadily increased in control. In contrast, the KMgHCA rats showed a decrease in SBP from baseline. A glucose tolerance test was administered in which 0.1 unit of regular insulin was injected along with glucose. At 7.5 minutes, there was a significantly lesser rise in glucose appearance in bloodstream. This finding indicates increased insulin sensitivity. (FIG. 6)

When losartan was injected, the SBP of both groups decreased. At 6 hours, the SBP were essentially the same. As shown in FIG. 7, the decreases in SBP's at 6 hours (−50±6.1 vs −21.7±7.0) were significantly different (p=0047). Thus, HCA appears to decrease angiotensin-2 in rats and to lower elevated SBP. Although insulin regulation likely is a factor in the blood pressure modulating effect of HCA, this evidence argues that inhibition of ACE is also important. Moreover, taken together with the evidence in Example 1, this second experiment helps to explain the difference in efficacy in blood pressure regulation between KCaHCA and the other HCA salts tested, to wit, although KCaHCA has little impact upon insulin metabolism, it nevertheless moderates blood pressure via ACE inhibition. Thus there is both direct and indirect evidence from experiments with several different salts of HCA indicating that the compound modulates ACE metabolism. ACE is known to be involved in vascular calcification.

### EXAMPLE 4

### Anti-Inflammatory: Effects Upon C-Reactive Protein and TNF-α

To test the properties of HCA in various forms under conditions similar to those found in human clinical trials, the inventor arranged for rats to be fed a diet in which 30% of the calories were obtained from fat under standard conditions, with a further approximately 20% of the calories being supplied as simple sugars. Such a dietary combination of fat and simple sugars is noted as promoting a variety of metabolic imbalances and dysfunctions. The rats were intubated twice daily with one of five HCA salts or placebo. On weekends, the HCA was added to the food at an approprate dosage. The amount of HCA in each arm of 8 animals was based on the minimum dosage which had been found effective in the form of the pure trisodium salt of HCA in tests by Hoffmann-La Roche in animals ingesting a 70% glucose diet, i.e., 0.33 mmoles/kg body weight HCA given twice per day. The HCA salts used were these: KCaHCA=a mixed potassium and calcium or double metal HCA salt commercially marketed as being entirely water soluble and of relatively high purity; KHCA=a relatively clean commercial potassium salt of HCA with a good mineral ligand attachment supplying 4467 mg potassium/100 grams of material; KMgHCA=three different dosage levels of an experimental potassium and magnesium salt with special characteristics, but suspected of being relatively unstable when exposed to stomach acid. The KCaHCA and KHCA salts were 60% HCA delivered at the rate of approximately 76 mg/day. The KMgHCA salts were delivered at the rate of 76 mg/day (r), 38 mg/day (l) and 228 mg/day (h), but due to initial miscalculations of the water of crystallization, this salt was only 45% HCA rather than 60%. The proper dosage for the KMgHCA(r) should have been 100 mg/day; the half dose (I) should have been 50 mg/day, and the triple dose (h) should have been 300 mg/day to match the commercial salts.

Tests were performed for C-reactive protein. Data was obtained for the animals at start and then at week 4 based on serum. Optical Density (OD) readings in the test kit used were 1 unit equals 50 picograms/mL. The delta changes over the 4 weeks for each arm vs control are shown.

| | | | Delta | | CRP |
|---|---|---|---|---|---|
| | Δ OD units | Standard | versus | Base- | Modu- |
| GROUP | after 4 wks | Error | Control | line | lation |
| Control | 339 | 113 | | | |
| KMgHCA(r) | −145 | 105 | 0.0007 | 0.0006 | ** |
| KMgHCA(l) | 33 | 70 | 0.0481 | 0.0268 | ** |
| KMgHCA(h) | −11.3 | 41 | 0.0186 | 0.0035 | ** |
| KHCA | −155 | 94 | 0.0005 | <0.0001 | ** |
| KCaHCA | 56 | 33 | 0.0756 | 0.0943 | |

** = significant

Four out of the five active arms showed significant improvements in the change (delta A) in CRP compared with control. In the cases of KMgHCA (r) and (h) as well as KHCA, the absolute readings for the arms also were lower at week 4 than initially, an interesting finding in that these were young animals and in rats, as in humans, inflammation tends to steadily increase over time, as was true in the control. Only the KCaHCA arm failed to yield significant results. The KCaHCA and the KMgHCA(l) arms were also the only two active arms in which absolute CRP levels increased, albeit only slightly.

In rats, blood pressure rises steadily with age, and this is what was seen in the control arm even over this short period of time. It should be noted that all active arms showed significantly lowered systolic blood pressure versus control at week 4 (data not shown). Similarly, by week 6, all the active arms had begun to diverge from control with lower body weights (data not shown), with the KHCA and the KCaHCA arms showing the greatest trend differences.

These results suggest that appetite regulation by HCA salts may not be controlled by or at least to the same extent by the same mechanisms with each particular salt as are other elements of the metabolism, such as inflammation. Even an extremely low dose of HCA as the KMgHCA salt used in this experiment had a stronger effect upon CRP levels than did the commercial KCaHCA salt used although the latter salt had a stronger effect upon weight gain. What is clear, however, is that several different HCA salts at different dosage levels positively modulated CRP in this experiment despite the short period of time allowed for results to appear.

At eight weeks, the findings were only slightly changed. With regard to CRP, readings at two months did not show statistical differences among the groups, although the means of all the test groups were lower than control. With regard to TNF-α, there was a trend toward a lowering in all groups compared to control. Using a simple t test versus control calculation in the case of TNF-α indicated significance with the low and intermediate doses of KMgHCA. Keeping in mind the small n, an increase in the number of test animals probably would have led to significance with regard to both CRP and TNF-α in all arms at eight weeks. Inflammation, especially that related to TNF-α, is known to play a role in vascular and other soft tissue calcification.

### EXAMPLE 5

### Leptin, Glucocorticoids, PPAR-γ and Resistin

OM rats aged 10 weeks to be fed a diet in which 30% of the calories were obtained from fat under standard conditions. The rats were intubated twice daily with one of three HCA salts or placebo. The amount of HCA in each arm of 5 animals was the minimum dosage which had been found effective in the form of the pure trisodium salt of HCA in tests by Hoffmann-La Roche in animals ingesting a 70% glucose diet, i.e., 0.33 mmoles/kg body weight HCA given twice per day. The HCA salts used were these: CaKHCA=a mixed calcium and potassium HCA salt commercially marketed as being entirely water soluble; KHCA 1=a relatively clean, but still hardly pure potassium salt of HCA with a good mineral ligand attachment supplying 44.67 grams potassium/100 grams of material; KHCA 2=an impure potassium salt of HCA with large amounts of gums attached and poor mineral ligand attachment supplying 21.69 grams potassium/100 grams of material. Data was collected with regard to serum insulin, leptin and cortisol levels.

| | Insulin | Leptin | Corticosterone |
|---|---|---|---|
| Group | ng/mL | ng/mL | ng/mL |
| Control | 2.655 | 9.52 | 269.38 |
| Control | 7.077 | 18.94 | 497.87 |
| Control | 4.280 | 34.34 | 265.71 |
| Control | 9.425 | 24.32 | 209.54 |
| Control | 3.798 | 8.40 | 116.12 |
| KHCA 1 | 3.880 | 9.93 | 45.79 |
| KHCA 1 | 4.399 | 7.31 | 33.10 |
| KHCA 1 | 3.181 | 9.25 | 65.57 |
| KHCA 1 | 3.210 | 24.36 | 55.40 |
| KHCA 1 | 3.639 | 9.07 | 84.62 |
| KHCA 2 | 4.427 | 9.13 | 26.02 |
| KHCA 2 | 4.301 | 9.75 | 270.83 |
| KHCA 2 | 3.245 | 8.00 | 45.44 |
| KHCA 2 | 3.695 | 9.16 | 45.63 |
| KHCA 2 | 2.053 | 8.26 | 38.04 |

Both of the potassium (−)-hydroxycitrate arms were superior to the calcium/potassium arm (data not shown here) in reducing insulin, leptin and corticosterone concentrations. Because of the difficulty in achieving significance with only 5 data points per arm, calculations regarding insulin and leptin combined the data from the two KHCA arms. With respect to insulin, the one-tailed P value was a significant 0.0306, and the two-tailed P value fell slightly short of significance at 0.0612. Using this combined data, there was also a significant one-tailed P value difference between the two KHCA arms and the result found with the CaKHCA. With respect to leptin, the two KHCA arms were combined, in part, because of one anomalously high data point and yielded a one-tailed P value which was a significant 0.0241 and a two-tailed P value which was significant at 0.0482. Corticosterone results were highly significant even at 5 data points per arm. KHCA 1 was easily significantly superior to control: the one-tailed P value was a highly significant 0.0048, and the two-tailed P value was a highly significant 0.0096.

Non-esterified fatty acid levels were not significantly different between control and the KHCA arms, but serum glucose and triglyceride levels exhibited a trend towards elevation. This is consistent with HCA's biophasic properties on a fatty diet and with published animal data to the effect that HCA elevates fatty acid oxidation at rest, although this effect is not significant during actual exercise. Elevated fatty acid oxidation typically slightly increases some fractions of blood fats, and also increases the rate of gluconeogenesis, hence may slightly increase blood glucose levels. However, in those individuals with markedly elevated blood glucose levels/glucose dysregulation, HCA can be used to improve glucose regulation. (U.S. Pat. No. 6,207,714) The same has been shown in animals with regard to elevated blood fats. The clear implication of these data is that HCA, if supplied in appropriate amounts, may be useful in reducing insulin levels and insulin resistance, leptin levels and leptin resistance, and elevated glucocorticoid levels. There was sustained reduction in weight gain found with KHCA 1 even after food consumption had returned to the level of control, a finding indicating an increased basal metabolic rate (BMR) and is in agreement with published studies already mentioned which give evidence of an increased BMR in HCA-treated animals.

It should be noted that an increased BMR is typical in cases in which fat consumption above the norm does not lead to weight gain. Elevated leptin blood levels have been found to correlate significantly in lean subjects with dietary fat intake and negatively with carbohydrate intake, whereas there is no correlation with total energy intake. Individuals who are lean on a chronically high fat diet (45% of calories) typically also have lower serum glucose levels. (Cooling J, Barth J, Blundell J. The high-fat phenotype: is leptin involved in the adaptive response to a high fat (high energy) diet? Int J Obes Relat Metab Disord. 1998 November;22(11): 1132-5.) This implies that some factor other than fatty acid oxidation, such as elevated insulin or glucocorticoid levels, has a role in inducing leptin resistance. Our findings suggest, based upon what is presently known of its actions, that the recently discovered signaling compound resistin likely is a common element involved in insulin resistance and leptin resistance which is affected by the chronic administration of adequate amounts of HCA. The impact of HCA upon resistin is itself mediated by way of peroxisome proliferator-activated receptor γ.

The evidence for this presently is indirect, yet a substantial case can be made. KHCA arms 1 and 2 significantly lowered insulin, leptin and glucocorticoid levels in comparison with control. This is important in that, as is true of insulin, in obese humans there is resistance to leptin and much elevated levels of leptin just as there is resistance to insulin and an elevated release of insulin. Elevated glucocorticoid levels increase leptin levels and may play a significant role in the development of leptin resistance, whereas norepinephrine and epinephrine decrease leptin production. (Fried S K, Ricci M R, Russell C D, Laferrere B. Regulation of leptin production in humans. J Nutr. 2000 December;130(12S Suppl):3127S-31S.) Long ago, it was observed that HCA incubated with white fat cells had an effect similar to that observed with epinephrine. (Fried S K, Lavau M, Pi-Sunyer F X. Role of fatty acid synthesis in the control of insulin-stimulated glucose utilization by rat adipocytes. J Lipid Res. 1981 July;22(5):753-62.)

Resistin levels are highly correlated with those of leptin. Resistin is exclusively made in adipose tissue. Moreover, its exclusive expression in adipocytes, its large increase during the late stage of adipogenesis, and its dramatic induction during fasting/refeeding and by insulin administration to streptozotocin-diabetic animals suggest that this factor may be involved in sensing the nutritional status of the animals to affect adipogenesis. Many of these properties are most similar to those observed with leptin, which is secreted only by adipocytes and is induced dramatically by fasting/refeeding and by diabetes/insulin. (Kee-Hong Kim, Kichoon Lee, Yang Soo Moon, and Hei Sook Sul. A Cysteine-rich Adipose Tissue-specific Secretory Factor Inhibits Adipocyte Differentiation. The Journal of Biological Chemistry 2001 April 6;276(14):11252-11256.) However, unlike resistin, leptin increases Krebs Cycle and uncoupling protein activity and it is an agonist for at least one peroxisome proliferator-activated receptor, that is, peroxisome proliferator-activated receptor a. (Ceddia R B, William W N Jr, Lima F B, Flandin P, Curi R, Giacobino J P. Leptin stimulates uncoupling protein-2 mRNA expression and Krebs cycle activity and inhibits lipid synthesis in isolated rat white adipocytes. Eur J Biochem. 2000 October;267(19):5952-8.)

The thiazolidinediones (TZDs), such as rosiglitazone, appear to work at least in part by down-regulating the expression of resistin while, and very likely by, up-regulating the actions of peroxisome proliferator-activated receptor-γ. As with resistin, the biological functions of PPAR-γ seem to be connected to fuel sensing. Agonists for the latter increase energy expenditure and reduce insulin resistance. Significantly, the TZDs also downregulate leptin gene expression, increase the flux through the Krebs Cycle and increase liver acetyl-CoA carboxylase, thus making cells more citrate-sensitive. As would be expected from this description, one side effect of rosiglitazone can be mild weight gain. (Thampy G K, Haas M J, Mooradian A D. Troglitazone stimulates acetyl-CoA carboxylase activity through a post-translational mechanism. Life Sci. 2000 December 29;68(6):699-708.) Rosiglitazone is thought to have no liver toxicity, but troglitazone, another TZD, certainly does.

The similarities between the actions of HCA and the TZDs is remarkable. HCA reduces insulin and leptin levels, increases the flux through the Krebs Cycle, increases liver acetyl-CoA carboxylase and, in at least one sense, makes cells more citrate-sensitive. The latter actions likely are those which activate PPAR-γ, for it has been shown elsewhere that an increase in long-chain CoA (acyl-CoA) affects the PPARs. (Belfiore F, lannello S. Insulin resistance in obesity: metabolic mechanisms and measurement methods. Mol Genet Metab. 1998 October;65(2): 121-8.) Activating PPAR-γ and reducing leptin levels, as already indicated, lowers resistin levels. (Steppan C M, Bailey S T, Bhat S, Brown E J, Banerjee R R, Wright C M, Patel H R, Ahima R S, Lazar M A. The hormone resistin links obesity to diabetes. Nature. 2001 January 18;409(6818):307-12.) Hence, in our view HCA provides the benefits and shares some of the primary mechanisms of action of the thiazolidinediones, but does not exhibit any of the toxicity found with some members of that class of drugs. When used properly, HCA not only does not promote the weight gain found with TZDs, it actually encourages weight loss. Therefore, HCA can be used to manipulate the resistin-PPAR-γ axis as well as the levels of insulin, leptin and glucocorticoids. As indicated in the text, all of these pathways have been shown to modulate vascular calcification.

### EXAMPLE 6

### A Standard Dosage Form

Numerous methods can be given as means of delivering HCA as required by the invention, including capsules, tablets, powders and liquid drinks. The following preparation will provide a stable and convenient dosage form.

| | | | | | 1 Kg |
|---|---|---|---|---|---|
| Ingredient | | Weight | | Percent | Batch |
| 1. | Aqueous Potassium | 500 | gm | 62.5% | 0.63 |
| | Hydroxycitrate | | | | |
| 2. | Calcium Carbonate | 50 | gm | 6.25% | 0.06 |
| 3. | Potassium Carbonate | 50 | gm | 6.25% | 0.06 |
| 4. | Anhydrous Lactose | 150 | gm | 18.75% | 0.19 |
| 5. | Cellulose Acetate Pthalate | 50 | gm | 6.25% | 0.06 |
| | Acetate | | | | |
| | Total | 800 | gm | 100.00% | 100.00 |

A. Blend items 1-5 in mixing bowl until smooth and even.

B. Take the liquid and spray into spray-drying oven at 300° C. until white powder forms. When powder has formed, blend with suitable bulking agent, if necessary, and compress into 800 mg tablets with hardness of 10-15 kg. This will mean that each tablet, if starting with 62% KHCA polymer powder, will have about 31% KHCA. However, if the tablets are pressed to 1600 mg, the dose will be equal to 800×62% KHCA.

C. After pressing the granulate through the screen, make sure that it flows well and compress into oblong tablets.

D. Tablets should have a weight of 1600 mg and a hardness of 14±3 kg fracture force. When tablets are completed, check for disintegration in pH 6.8, 0.05M KH2PO4. Disintegration should occur slowly over 4-5 hours.

### EXAMPLE 7

### An Enteric Softgel Dosage Form

Soft gelatin encapsulation is used for oral administration of drugs in liquid form. For this purpose, HCA may be provided in a liquid form by suspending it in oils, polyethylene glycol-400, other polyethylene glycols, poloxamers, glycol esters, and acetylated monoglycerides of various molecular weights adjusted such as to insure homogeneity of the capsule contents throughout the batch and to insure good flow characteristics of the liquid during encapsulation. The soft gelatin shell used to encapsulate the HCA suspension is formulated to impart enteric characteristics to the capsule to ensure that the capsule does not disintegrate until it has reached the small intestine. The basic ingredients of the shell are gelatin, one or more of the enteric materials listed above, plasticizer, and water. Care must be exercised in the case of softgels to use the less hygroscopic salts and forms of HCA or to pretreat the more hygroscopic salts to reduce this characteristic. The carrier may need to be adjusted depending on the HCA salt, ester or amide used so as to avoid binding of the ingredients to the carrier. Water should never be used as a carrier. Various amounts of one or more plasticizer are added to obtain the desired degree of plasticity and to prevent the shell from becoming too brittle.

### EXAMPLE 8

A CONTROLLED-DELIVERY DOSAGE FORM

| | | |
|---|---|---|
| Ingredient | mg/Tablet | Percent |

| | | | | |
|---|---|---|---|---|
| 1. | HCA calcium salt | 500.00 | mg | 71.43% |
| 2. | Microcrystalline cellulose | 17.00 | mg | 2.42% |
| 3. | Dicalcium phosphate | 45.00 | mg | 6.42% |
| 4. | Corn starch | 9.00 | mg | 1.28% |
| 5. | TPGS | 46.00 | mg | 6.60% |
| 6. | Hydrogenated vegetable oil | 50.00 | mg | 7.14% |
| 7. | Cellulose acetate phthalate | 15.00 | mg | 2.14% |
| 8. | Carbopol ® 974P Carbomer | 15.00 | mg | 2.14% |
| 9. | Magnesium Sterate | 3.00 | mg | 0.43% |
| | TOTAL | 700.00 | mg | 100.00% |

1. Weigh and blend items 1-4 in a fluid bed dryer and blend for 4-5 minutes. Dissolve item #5 by heating to 40° C. until molten then stir with magnetic stir rod. After the powders are blended, continue steady blending while adding the TPGS as a molten liquid. Pour in all fluid until an even granulate is formed. Next melt the hydrogenated vegetable oil until molten and fluid in nature. Spray this material at the same time stirring with a magnetic stir rod. Continue blending with air at 30° C. When all the material is thoroughly coated and the granulate is hardened, spray the cellulose acetate phthalate which has been completely dissolved in ammoniated water. Continue spraying until all the granulate has been covered then allow to dry at room temperature in the fluid bed dryer with continuous blending. Remove the granulate from the bowl, when the granulate is dry, pass through an #093 screen using a D3 Fitzmill comminutor.

2. When the granulate has been dried and reduced in size, blend in fluid bed first with Carbopol_{—}974P, then when completely blended, add magnesium stearate and blend for 2-3 minutes.

3. Place the mixed granulate on a rotary press and compress the material into tablets with a weight of 700 mg and a fracture force of 10-15 kg.

### CONCLUSIONS

(−)-Hydroxycitrate has a multitude of metabolic functions. The literature teaches that the compound reduces blood lipids, induces weight loss and decreases appetite in both animals and humans. However, the inventor has discovered that this compound can be employed for reducing and regulating calcification of the blood vessels and other soft tissues. Such regulation offers benefits against arterial calcification and vascular diseases, osteoarthritis, rheumatoid arthritis, and the calcification of surgical stints, such as those containing elastin. This safe use for ameliorating problems of soft tissue calcification is an entirely unexpected and novel employment of (−)-hydroxycitric acid, its derivatives and its salt forms.

I claim:

1. A method for preventing, treating or ameliorating vascular and soft tissue calcification and their symptoms in an individual in need thereof which is comprised of administering orally an effective amount of (−)-hydroxycitric acid.

2. The method of claim 1 where the (−)-hydroxycitric acid is supplied in a therapeutically effective amount of the free acid or its lactone.

3. The method of claim 1 where the (−)-hydroxycitric acid is supplied in a therapeutically effective amount of the alkali metal salts potassium or sodium (−)-hydroxycitrate.

4. The method of claim 1 where the (−)-hydroxycitric acid is supplied in a therapeutically effective amount of the alkaline earth metal salts calcium or magnesium (−)-hydroxycitrate.

5. The method of claim 1 where the (−)-hydroxycitric acid is supplied in a therapeutically effective amount of a mixture the alkali metal salts and/or the alkaline earth metal salts of (−)-hydroxycitrate or some mixture of alkali metal salts and alkaline earth metal salts of (−)-hydroxycitrate or in the form of therapeutically effective amide and/or ester derivatives of (−)-hydroxycitric acid.

6. The method of claim 1 where the (−)-hydroxycitric acid is supplied in a therapeutically effective amount as the free acid, its lactone or as one or more of the salts or other derivatives of the free acid and is delivered in a controlled release form.
